(19) United States
(12) Patent Application Publication
(10) Pub. No.: US 2025/0195711 A1
(43) Pub. Date: Jun. 19, 2025

(54) DELIVERY ENGINE AND APPARATUS FOR DELIVERING A VOLATILE MATERIAL

(71) Applicant: The Procter & Gamble Company, Cincinnati, OH (US)

(72) Inventors: Rahul VYAS, Singapore (SG); Bhavesh RAJWANI, Singapore (SG); Walter SORDO, Trento (IT); Stefano BALDESSARI, Caldonazzo (IT)

(21) Appl. No.: 18/983,737

(22) Filed: Dec. 17, 2024

Related U.S. Application Data
(60) Provisional application No. 63/611,920, filed on Dec. 19, 2023.

(51) Int. Cl. A61L 9/12

(52) U.S. Cl. CPC A61L 9/127; A61L 2209/131; A61L 2209/133

(57) ABSTRACT

A delivery engine for delivering a volatile material, and an apparatus comprising the delivery engine are provided. The delivery engine may include a primary reservoir portion containing an initial volume of volatile material, where the volatile material is configured to evaporate from the primary reservoir portion after an activation step is performed, and at least one secondary reservoir portion. When the delivery engine is oriented for use, at least one secondary reservoir portion is gravitationally below the primary reservoir portion and a total volume of the at least one secondary reservoir portion that is gravitationally below the primary reservoir portion is above a certain threshold.

### FIELD OF THE INVENTION

The present invention relates to a delivery engine for delivering a volatile material, particularly to a delivery engine for delivering a volatile material to the environment within an enclosed space, such as a room or vehicle.

### BACKGROUND OF THE INVENTION

It is generally known to use a device to evaporate a volatile material into a space, particularly a domestic space, in order to deliver a variety of benefits, such as air freshening or perfuming of the air. Non-energized systems, for example, systems that are not powered by electrical energy, are a popular way for the delivery of volatile materials into the atmosphere.

Examples of non-energised systems include membrane-based systems such as those disclosed in PCT patent publications WO2017/192638, WO2017/192639, WO2017/192640, and WO2017/192641. While such systems have enjoyed major commercial success, there remains the possibility of improvement. For example, such systems include a window on the front of the housing through which a liquid perfume composition may be visible. This may provide aesthetic benefits, especially when a coloured perfume composition is used, as well as allowing a user to easily determine how much perfume composition remains in the apparatus. However, such products typically reach the end of their effective life before the perfume composition has completely evaporated. In addition, perfume compositions often contain a small quantity of non-volatile components that will remain at the end of the product's effective life. This leads to a small amount, for example around 10% of the initial volume, of liquid remaining in the apparatus at end-of-life. However, the visibility of this remaining perfume composition causes confusion with end users, leading to a belief that the product still contains perfume and has not reached end-of-life. This leads to consumers retaining the products beyond end of life, and resulting dissatisfaction with perfume performance.

There is a need for an apparatus for delivering a volatile material which addresses at least some of the drawbacks associated with the prior art and causes reduced consumer confusion.

### SUMMARY OF THE INVENTION

The present invention addresses one or more of the drawbacks associated with the prior art. By providing a delivery engine comprising at least two reservoir portions, with a first reservoir portion intended to be visible through the front of a housing of an apparatus as described herein, and at least a second reservoir portion that is not intended to be visible through the front of a housing of the apparatus, it is possible to ensure that volatile material remaining in an apparatus at end-of-life is not visible to a consumer, reducing confusion.

Therefore, the present invention provides the following.
1. A delivery engine for delivering a volatile material, the delivery engine comprising:
   a primary reservoir portion containing an initial volume of volatile material, where the volatile material is configured to evaporate from the primary reservoir portion after an activation step is performed; and
   at least one secondary reservoir portion;
wherein when the delivery engine is oriented for use:
at least one secondary reservoir portion is gravitationally below the primary reservoir portion; and
a total volume of the at least one secondary reservoir portion that is gravitationally below the primary reservoir portion is at least 4% of the initial volume of volatile material.
2. The delivery engine according to clause 1, wherein when the delivery engine is oriented for use, a total volume of the at least one secondary reservoir portion that is gravitationally below the primary reservoir portion is from 4% to 30% of the initial volume of volatile material, optionally from 5% to 20% of the initial volume of volatile material;
more optionally from 7% to 15% of the initial volume of volatile material.
3. The delivery engine according to clause 1 or 2, wherein either (a) or (b) applies:
(a) the primary reservoir portion and at least one secondary reservoir portion are in fluid communication, or
(b) the delivery engine is configured such that after an activation step is performed, the primary reservoir portion and at least one secondary reservoir portion are in fluid communication.
4. The delivery engine according to clause 3, wherein when:
the delivery engine is oriented such that at least one secondary reservoir portion is gravitationally below the primary reservoir portion, and
the primary reservoir portion is in fluid communication with at least one secondary reservoir portion,
then the at least one secondary reservoir portion is able to hold a volume of volatile material that is at least 4% of the initial volume of volatile material in a state that is gravitationally below the primary reservoir.
5. The delivery engine according to any one of the preceding clauses, wherein the delivery engine is a single-use delivery engine and/or wherein the delivery engine is not refillable.
6. The delivery engine according to any one of the preceding clauses, wherein the volatile material is configured to simultaneously evaporate from both the primary reservoir portion and at least one secondary reservoir portion after an activation step is performed.
7. The delivery engine according to any one of the preceding clauses, wherein the delivery engine comprises a microporous membrane enclosing the primary and secondary reservoir portions, where the microporous membrane is configured to allow evaporation of volatile material contacting the microporous membrane.
8. The delivery engine according to clause 7, wherein after an activation step is performed, the microporous membrane is configured to allow evaporation of volatile material present in both the primary reservoir portion and at least one secondary reservoir portion.
9. The delivery engine according to any one of the preceding clauses, wherein the delivery engine is for use in a housing, and the primary reservoir portion is configured to be received by a window portion of the housing.
10. The delivery engine according to any one of the preceding clauses, wherein the primary reservoir portion is formed from a transparent or translucent material.
11. The delivery engine according to any one of the preceding clauses, wherein the delivery engine is for use in a housing, and one or both of (i) and (ii) apply:
   (i) the at least one secondary reservoir portion is configured to be at least partially obscured by the housing;
   (ii) the at least one secondary reservoir portion is formed from an opaque material.
12. The delivery engine according to any one of the preceding clauses, wherein the delivery engine comprises at least one secondary reservoir portion at each of two opposing ends of the primary reservoir portion.
13. The delivery engine according to any one of the preceding clauses, wherein the delivery engine is configured to release a volatile material to a surrounding environment without an artificially generated airflow.
14. A delivery engine for delivering a volatile material, the delivery engine comprising:
   a primary reservoir portion containing a volatile material, where the volatile material is configured to evaporate from the primary reservoir portion after an activation step is performed; and
   at least one secondary reservoir portion;
   wherein when the delivery engine is oriented for use:
   at least one secondary reservoir portion is gravitationally below the primary reservoir portion; and
   a total volume of the at least one secondary reservoir portion that is gravitationally below the primary reservoir portion is at least 0.24 mL, optionally at least 0.3 mL, more optionally at least 0.5 mL.
15. An apparatus for delivering a volatile composition, the apparatus comprising:
a housing having a front part, a back part; and
a delivery engine located within the housing, the delivery engine comprising:
a primary reservoir portion containing a volatile material, where the volatile material is configured to evaporate from the primary reservoir portion or where the volatile material is configured to evaporate from the primary reservoir portion after an activation step is performed; and
   at least one secondary reservoir portion;
wherein the housing is configured such that when the apparatus is oriented for use:
   at least one secondary reservoir portion of the delivery engine is gravitationally below the primary reservoir portion of the delivery engine;
the primary reservoir portion of the delivery engine is visible through the front part of the housing, and one or both of (i) and (ii) apply:
(i) the at least one secondary reservoir portion that is gravitationally below the primary reservoir portion is at least partially obscured by the front part of the housing;
(ii) the at least one secondary reservoir portion that is gravitationally below the primary reservoir portion is formed from an opaque material.
16. The apparatus according to clause 15, wherein the back part of the housing comprises a plurality of apertures for exposing an evaporative portion of the delivery engine,
   optionally wherein the delivery engine comprises a microporous membrane enclosing the primary and secondary reservoir portions, where the microporous membrane is configured to allow evaporation of liquid volatile material, and where the evaporative portion of the delivery engine is the microporous membrane.
17. The apparatus according to clause 15 or 16, wherein the housing is openable and the delivery engine is replaceable,
optionally wherein the housing comprises a releasable locking mechanism for releasably closing the housing.
18. The apparatus according to any one of clauses 15 to 17, wherein when the apparatus is oriented for use a total volume of the at least one secondary reservoir portions that are gravitationally below the primary reservoir portion is at least 4% of an initial volume of volatile material.

### BRIEF DESCRIPTION OF THE DRAWINGS

FIGS. 1A and 1B show two views of a delivery engine according to the invention.

FIG. 2 shows two further views of the delivery engine of FIG. 1.

FIG. 3 shows an alternative delivery engine according to the invention.

FIG. 4 shows a rear view of a delivery engine according to the invention.

FIGS. 5A and 5B show two views of an apparatus according to the invention, which apparatus comprises a delivery engine disclosed herein.

### DETAILED DESCRIPTION OF THE INVENTION

The invention provides a delivery engine for delivering a volatile material, the delivery engine comprising:
a primary reservoir portion containing an initial volume of volatile material, where the volatile material is configured to evaporate from the primary reservoir portion after an activation step is performed; and
at least one secondary reservoir portion;
wherein when the delivery engine is oriented for use:
at least one secondary reservoir portion is gravitationally below the primary reservoir portion; and
a total volume of the at least one secondary reservoir portion that is gravitationally below the primary reservoir portion is at least 4% of the initial volume of volatile material.

As used herein, the word “comprising” may be interpreted as requiring the features mentioned, but not limiting the presence of other features. Alternatively, the word “comprising” may also relate to the situation where only the components/features listed are intended to be present (e.g., the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material containing minor amounts of other chemicals, which other chemicals do not materially change the properties of the material in question. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

As used in this specification and the appended claims, the singular forms “a”, “an”, and “the” include plural references unless the content clearly dictates otherwise. Thus, for example, “a volatile material” may include more than one volatile material.

As used herein, the term “delivery engine” means a part (e.g., a part of an air freshening apparatus) that is capable of delivering a volatile material to the surrounding atmosphere. The delivery engine may be a replaceable/consumable part of an apparatus, or may be provided as a part of a single-use disposable apparatus.

Typically, the delivery engine is a single-use delivery engine and is not refillable. Thus, after evaporation of the volatile material from the delivery engine, the delivery engine may be intended to be disposed of, rather than refilled with volatile material.

Preferably, the invention relates to delivery engine for a non-energized apparatus for the delivery of a volatile material to the atmosphere in a continuous, non-energized manner. “Non-energized” means that the apparatus is passive does not require to be powered by a source of external energy. In particular, the apparatus does not need to be powered by a source of heat, gas, or electrical current, and the volatile material is not delivered by aerosol means.

The delivery engine of the present invention is able to deliver a volatile material in a substantially continuous manner when the apparatus is in a resting position (i.e., the apparatus is not being moved). The emission level of volatile materials may exhibit a substantially uniform intensity until substantially all the volatile materials are exhausted. The continuous emission of the volatile materials can be of any suitable length, including but not limited to, up to: 20 days, 30 days, 60 days, 90 days, shorter or longer periods, or any period between 30 to 90 days, such as about 8 weeks (56 days).

The delivery engine of the present invention is suitable for purposes of providing fragrances, air fresheners, deodorizers, odor eliminators, malodor counteractants, insecticides, insect repellents, medicinal substances, disinfectants, sanitizers, mood enhancers, and aromatherapy aids, or for any other purpose using a volatile material that acts to condition, modify, or otherwise change the atmosphere or the environment. For purposes of illustrating the present invention in detail, but without intending to limit the scope of the invention, the invention will be described in the context of an air freshening system for delivering liquid containing perfume raw materials.

### Primary and Secondary Reservoir Portions

The delivery engine comprises a primary reservoir portion and at least one secondary reservoir portion. As used herein, a “reservoir portion” is a distinct portion or part of a reservoir of the delivery engine. The primary and secondary reservoir portions may be connected such that they are in fluid (e.g., liquid) communication. Alternatively, they may be provided in a fluidly isolated state, where the delivery engine is activatable such that after activation, the primary and secondary reservoir portions may be in fluid (e.g., liquid) communication.

As discussed herein, the volatile material is configured to evaporate from the primary reservoir portion after an activation step is performed. In this context, evaporation from the primary reservoir portion is to be understood as meaning that volatile material located within the primary reservoir portion may evaporate from the primary reservoir portion without needing to first pass to a secondary reservoir portion. For example, as discussed in more detail herein, volatile material may evaporate from the primary reservoir portion through a microporous membrane that encloses the primary reservoir portion. In some embodiments of the invention, the volatile material may be configured to simultaneously evaporate from both the primary reservoir portion and at least one secondary reservoir portion, such as through one or more microporous membranes enclosing the primary reservoir portion and at least one secondary reservoir portion.

While the invention is generally described herein in relation to the volatile material being configured to evaporate from the primary reservoir portion after an activation step, a skilled person will appreciate that equivalent delivery engines may be provided in which the volatile material is configured to evaporate from the delivery engine in general.

Therefore, also provided herein is a delivery engine for delivering a volatile material, the delivery engine comprising:
a primary reservoir portion containing an initial volume of volatile material, where the volatile material is configured to evaporate from the delivery engine after an activation step is performed; and
at least one secondary reservoir portion;
   wherein when the delivery engine is oriented for use:
at least one secondary reservoir portion is gravitationally below the primary reservoir portion; and
a total volume of the at least one secondary reservoir portion that is gravitationally below the primary reservoir portion is at least 4% of the initial volume of volatile material.

Any appropriate features of the invention described herein may apply equally to the above delivery engine.

The primary reservoir portion has a greater volume than each of the secondary reservoir portion(s), such that an initial volume of volatile material is greater than the volume of each of the secondary reservoir portions. Typically, the volume of the primary reservoir portion, and the initial volume of volatile material, are each greater than the cumulative volume of the secondary reservoir portion(s). In any event, the volume of the primary reservoir portion, and the initial volume of volatile material, are each greater than the cumulative volume of secondary reservoir portion(s) that may be simultaneously gravitationally below the primary reservoir portion. For example, the volume of the primary reservoir portion, and/or the initial volume of volatile material, may be at least 1.5 times greater than, at least 2 times greater than, at least 3 times greater than, at least 4 times greater than, at least 5 times greater than, at least 6 times greater than, at least 7 times greater than, at least 8 times greater than, at least 9 times greater than, or at least 10 times greater than, the cumulative volume of secondary reservoir portion(s) that may be simultaneously gravitationally below the primary reservoir portion.

The invention is described herein in relation to a delivery engine in which the primary reservoir portion contains an initial volume of volatile material. However, person skilled in the art will appreciate that a part of the initial volume of volatile material may be present in the at least one secondary reservoir portion. Therefore, in some embodiments of the invention, the delivery engine contains an initial volume of volatile material, which is contained within the primary, and optionally secondary, reservoir portions. In such cases, the at least one secondary reservoir portion that is gravitationally below the primary reservoir portion has a volume that is at least 4% of the initial volume of volatile material held within the delivery engine as a whole.

The initial volume of volatile material refers to an amount of volatile material that is present within the delivery engine before an activation step is conducted. Thus, in some embodiments of the invention, the delivery engine is an activatable delivery engine and the initial volume of volatile material is the amount of volatile material present before activation. As explained herein, the delivery engine is configured such that the volatile material may evaporate from the delivery engine after an activation step is performed. Therefore, the initial volume of volatile material may refer to the amount of volatile material present in a delivery engine at the point of purchase by a consumer, before the delivery engine is activated to allow evaporation of the volatile material.

The primary and secondary reservoir portion(s) may be directly connected (or directly connectable by an activation step). Alternatively, the primary and secondary reservoir portion(s) may be connected (or connectable by an activation step) by one or more conduits running from the primary reservoir portion to the secondary reservoir portion(s). In such cases, a conduit may run from the primary reservoir portion to a single secondary reservoir portion, or to multiple secondary reservoir portions. Therefore, the invention includes the case where multiple secondary reservoir portions are connected to the primary reservoir portion by separate conduits, and the case where multiple secondary reservoir portions are connected to the primary reservoir portion by a single conduit. As discussed herein, a benefit of the invention is that when a residual amount of volatile material remains in the delivery engine, it may be held within at least one secondary reservoir portion that is gravitationally below the primary reservoir portion during use. As a result, the residual volatile material is (substantially) not visible within the primary reservoir portion. In order to provide this benefit, the at least one secondary reservoir portion that is gravitationally below the primary reservoir portion has a volume that is at least 4% of the initial volume of volatile material. For the purposes of calculating this percentage, the volume of such secondary reservoir portion(s) is taken to include the volume of any conduit that is gravitationally below the primary reservoir portion and connects the primary reservoir portion to the secondary reservoir portion. Therefore, the term “secondary reservoir portion” used herein may be understood as including any conduit that connects the primary reservoir portion to the secondary reservoir portion.

For the avoidance of doubt, the invention enables the residual volatile material to be partially or totally held within secondary reservoir portions, reducing or eliminating the presence of residual volatile material in the primary reservoir portion at the end of life. This more clearly signals to a user that the product has reached end of life, and this benefit is obtained even if the residual volatile material is not completely held in the secondary reservoir portion, because the amount of residual volatile material visible in the primary reservoir portion will be significantly reduced. By way of example, if a volume of the secondary reservoir portion is 4% of the initial volume of volatile material, and the amount of residual volatile material is greater than 4% of the initial volume (e.g., 10%), the invention will still significantly, and advantageously, reduce the amount of residual volatile material left in the primary reservoir portion, and visible to a user, at end of life.

The primary reservoir portion contains a volatile material. The at least one secondary reservoir portion may also contain the volatile material, or the secondary reservoir portion may be provided without volatile material until an activation of the delivery engine is performed, upon which volatile material may flow or otherwise pass into one or more secondary reservoir portions. Thus, in some embodiments the at least one secondary reservoir portion may contain a volatile material, which may be the same as or different to that contained in the primary reservoir portion. In other embodiments, the at least one secondary reservoir portion may not contain the volatile material, but the delivery engine may be configured such that after an activation, the volatile material may flow into at least one secondary reservoir portion.

As described herein, when the delivery engine is oriented for use:
at least one secondary reservoir portion is gravitationally below the primary reservoir portion; and
a total volume of the at least one secondary reservoir portion that is gravitationally below the primary reservoir portion is at least 4% of the initial volume of volatile material.

As used herein, “oriented for use” refers to the orientation that a delivery engine, apparatus or housing might reasonably be placed in during use by a consumer. For example, if an apparatus comprises a base, then oriented for use would refer to the orientation when the base is placed on a substantially flat surface. If an apparatus comprises a hook, then oriented for use would refer to the orientation resulting from the apparatus being suspended from said hook. In the context of a delivery engine, “oriented for use” means the orientation that the delivery engine would have when placed inside a housing or apparatus suitable for receiving the delivery engine, and the housing or apparatus is oriented for use as described above. If a delivery engine, apparatus or housing is provided as part of a package or kit with instructions for use, then “oriented for use” may refer to the orientation that would result from following the instructions for use.

As used herein, and provided the first and second reservoir portions are fluidly connected, when a secondary reservoir portion is “gravitationally below” the primary reservoir portion, then the secondary reservoir portion is positioned such that liquid present in the primary reservoir portion is able to flow under the force of gravity to the secondary reservoir portion. The liquid may flow through a connection (e.g., aperture) directly from the primary reservoir portion to the secondary reservoir portion, or via a conduit connecting the primary and secondary reservoir portions. When at least one secondary reservoir portion is gravitationally below the primary reservoir portion, a total volume of the at least one secondary reservoir portion that is gravitationally below the primary reservoir portion is at least 4% of the initial volume of volatile material. In the case where the secondary reservoir portion(s) include a part that is below the primary reservoir portion and a part that is not below the primary reservoir portion, the volume of the part that is below the primary reservoir portion is at least 4% of the initial volume of volatile material. Thus, when the delivery engine according to the invention contains less than 4% of the initial volume of volatile material and is oriented in this way, substantially all of the remaining volatile material is held in a secondary reservoir portion, and the primary reservoir portion is substantially empty. A person skilled in the art will appreciate that a small amount (e.g., droplets) of volatile material may remain in the primary reservoir portion.

In some embodiments of the invention, when the delivery engine is oriented for use, a total volume of the at least one secondary reservoir portion that is gravitationally below the primary reservoir portion may be from 4% to 30% (e.g., from 5% to 20%, such as from 7% to 15%) of the initial volume of volatile material.

For the avoidance of doubt, any end point of any range defined herein may be combined with any other end point of any other range in respect of the same variable. Therefore, for the percentage ranges above, the following are explicitly disclosed:
from 4% to 5%, from 4% to 7%, from 4% to 15%, from 4% to 20%, from 4% to 30%;
from 5% to 7%, from 5% to 15%, from 5% to 20%, from 5% to 30%;
from 7% to 15%, from 7% to 20%, from 7% to 30%;
from 15% to 20%, from 15% to 30%; and
from 20% to 30%.

Purely by way of example, when the delivery engine is oriented for use, a total volume of the at least one secondary reservoir portion that is gravitationally below the primary reservoir portion may be at least 0.24 mL, such as at least 0.3 mL or at least 0.5 mL. These minimum volumes may be appropriate for a delivery engine that contains from about 6 mL to about 10 mL of volatile material.

The amount of volatile material that remains in the delivery engine at end of life is not particularly limited, and the invention will provide the beneficially reduced amount of residual volatile material in the primary reservoir irrespective of the absolute amount of volatile material remaining within the delivery engine. Nevertheless, it may be desirable for the delivery engine to release or evaporate substantially all of the volatile material during the delivery engine's lifetime, which as mentioned above may be any appropriate duration such including but not limited to, up to: 20 days, 30 days, 60 days, 90 days, shorter or longer periods, or any period between 30 to 90 days, such as about 8 weeks (56 days). In this context, “substantially all of the volatile material” may refer to at least 75%, at least 80%, at least 85%, at least 90%, or at least 95% of the volatile material during the delivery engine's intended lifetime. Purely by way of example, the delivery engine may release at least 85% (preferably at least 90%) of the volatile material during 8 weeks at 23° C. and 45% relative humidity in a room having a volume of 49 m³, and an average air intake/exhaust of from of 2.9 m³/min to 4.2 m³/min.

This configuration of the delivery engine of the invention advantageously means that, where the primary reservoir portion is visible to a user but the secondary reservoir portion(s) are not, the delivery engine appears empty despite the presence of a small amount of unevaporated volatile material within the delivery engine. This helps to avoid user confusion at the end of the delivery engine's effective lifetime.

The delivery engine of the invention may comprise at least one secondary reservoir portion at each of two opposing ends of the primary reservoir portion. For example, the delivery engine may comprise at least one secondary reservoir portion at each of two ends of the delivery engine (e.g., in a longitudinal direction), with the primary reservoir portion being located between the secondary reservoir portions. This configuration advantageously means that there may always be at least one secondary reservoir portion gravitationally below the primary reservoir portion. In addition, a delivery engine comprising at least one secondary reservoir portion at each of two opposing ends of the primary reservoir portion may be placed into a housing in more than one way (e.g., in two opposite directions), ensuring that that at least one secondary reservoir portion is gravitationally below the primary reservoir portion irrespective of the direction of the delivery engine within the housing. Thus, the delivery engine may have multiple independent orientations for use that differ from each other by at least 30°, at least 45°, at least 90° or at least 180° (such as about 30°, about 45°, about 90° or about) 180°, where each orientation provides a different secondary reservoir portion gravitationally below the primary reservoir portion, or a different combination of secondary reservoir portions gravitationally below the primary reservoir portion.

Such configurations advantageously improve ease of use for a consumer, whilst also maintaining aesthetic benefits (e.g., symmetry) when compared to delivery engines that may only be inserted in one way.

The primary and secondary reservoir portions may be made from any appropriate material. Typically, the primary reservoir portion is made from a transparent or translucent thermoplastic polymer to enable a user to easily view the fill level of the primary reservoir portion. For the avoidance of doubt, when used in this context, “transparent or translucent” includes any material that has both transparent/translucent portions, and opaque portions, provided that the transparent/translucent portions enable a user to view the fill level of the primary reservoir portion.

From the perspective of ease of manufacture, it may be desirable for the one or more secondary reservoir portions to be made from a transparent or translucent thermoplastic polymer. However, the one or more secondary reservoir portions may also be formed from an opaque material, such as an opaque thermoplastic polymer.

For the avoidance of doubt, while the invention is described herein in relation to a delivery engine comprising a single primary reservoir portion, and associated secondary reservoir portions, a person skilled in the art will appreciate that the invention may be applied to delivery engines that comprise two or more different primary reservoir portions, each having one or more associated secondary reservoir portions as described herein. For example, a delivery engine may comprise a first primary reservoir portion containing a first volatile material, and a second primary reservoir portion containing a second volatile material. Each of the first and second primary reservoir portions may have one or more associated secondary reservoir portions as described herein. The use of two or more primary reservoir portions, each containing different volatile materials, may allow for a more controlled evaporation of the different volatile materials than if all volatile materials are present in a single primary reservoir portion.

For brevity, the term “reservoir” may be used hereinbelow to refer collectively to the primary and secondary reservoir portions.

### Microporous Membrane

The delivery engine may comprise a microporous membrane, which for the sake of brevity may be referred to herein as “the membrane”. The microporous membrane may enclose the reservoir (i.e., the primary reservoir portion and the at least one secondary reservoir portions) such that volatile material is unable to escape from the delivery engine without passing through the microporous membrane. The microporous membrane may prevent the passage of liquid, such that the volatile material is only able to escape the delivery engine by evaporating through, or from, the microporous membrane.

The microporous membrane is vapor permeable and capable of wicking liquid, yet prevents free flow of liquid out of the microporous membrane. The microporous membrane may have a volume average pore diameter of from 0.065 μm to 0.15 μm. The use of a microporous membrane having such a pore size provides a number of advantages as discussed herein.

Without being bound by theory, it is believed that microporous membranes having a volume average pore diameter of less than 0.065 μm provides inferior perfume release. It is also believed that microporous membranes having higher volume average pore diameters may suffer from leaking and/or sweating. In some embodiments, the microporous membrane may have a volume average pore diameter of from 0.07 to 0.12 μm, such as from 0.07 to 0.11 μm, or 0.08 to 0.1 μm.

Typically, the microporous membrane has a pore size distribution such that at least 50%, such as at least 60%, such as at least 70%, such as at least 80% or such as at least 90% of the pores of the microporous membrane have a pore diameter of from 0.065 μm to 0.15 μm.

The microporous membrane may comprise (e.g., be formed from) polyethylene, such as ultra-high molecular weight polyethylene (UHMWPE), though other length polyethylene chains may also be used. As used herein, UHMWPE refers to polyethylene having a molecular mass of from about 3.5 million to 7.5 million amu.

The microporous membrane may have a thickness in the z-direction, of about 0.01 mm to about 1 mm, alternatively between about 0.2 mm to about 0.4 mm, from about 0.22 to about 0.37 mm, e.g., from about 0.25 to about 0.35 mm.

The microporous membrane may be formed from a single piece, or single sheet, of material. In other words, the microporous membrane may not be laminated. Thus, the microporous membrane may be formed from a single sheet of polyethylene having a thickness as described above.

Those of ordinary skill in the art will appreciate that the surface area of the microporous membrane can vary depending on the user preferred size of the delivery engine. In some embodiments, the (evaporative) surface area of the microporous membrane may be about 2 cm² to about 100 cm², alternatively about 10 cm² to about 50 cm², alternatively about 10 cm² to about 45 cm², alternatively about 10 cm² to about 35 cm², alternatively about 15 cm² to about 40 cm², alternatively about 15 cm² to about 35 cm², alternatively about 20 cm² to about 35 cm², alternatively about 30 cm² to about 35 cm², alternatively about 35 cm².

The microporous membrane may have any appropriate porosity. For example, the microporous membrane may have a porosity of from 45% to 70%, on a volume basis, such as from 45% to 65%. In certain embodiments of the invention, the porosity may be from 50 to 70%, such as 55 to 65%.

The microporous membrane may have any appropriate total pore volume, such as from 0.6 to 2 cm³/g. Typically, the total pore volume may be from 0.65 to 1.6 cm³/g, such as 0.7 to 1.5 cm³/g. In certain embodiments of the invention, the total pore volume may be from 0.8 to 1.4 cm³/g.

The microporous membrane may have any appropriate bulk density, such as from 0.3 to 0.8 g/cm³. Typically, the bulk density may be from 0.35 to 0.75 g/cm³, such as from 0.4 to 0.7 g/cm³. In certain embodiments of the invention, the bulk density may be from 0.4 to 0.6 g/cm³.

Suitable microporous membranes for the present invention include microporous polyethylene membranes having the properties described herein, available from Microporous, LLC.

The microporous membrane may comprise any suitable filler and plasticizer known in the art. Fillers may include finely divided silica, clays, zeolites, carbonates, charcoals, and mixtures thereof. In one embodiment, the microporous membrane may be filled with about 30% to about 80%, by total weight, of silica.

In one aspect of the invention, the microporous membrane may include a dye that is sensitive to the amount of volatile material it is in contact with to indicate end-of-life. Alternatively, the microporous membrane may change to transparent when in contact with a fragrance or volatile material to indicate diffusion is occurring. Other means for indicating end-of-life that are known in the art are contemplated for the present invention.

The membranes described herein may advantageously provide a clear visual change when wetted with volatile material, and when dry (whether before use or at end of life). Such visual changes may be more detectible when the membrane does not comprise a white pigment (e.g., TiO₂). Therefore, the microporous membrane may comprise less than 5 wt. % of a white pigment, such as less than 1 wt. % of a white pigment, less than 0.1 wt. % of a white pigment, or less than 0.01 wt. % of a white pigment. The microporous membrane may be free from a white pigment.

The visual change when the membrane is wetted as compared to dry may be more noticeable when the microporous membrane comprises a coloured dye/pigment or a black dye/pigment. Therefore, the microporous membrane may comprise a coloured or black dye/pigment, such as activated charcoal. Such a coloured or black pigment/dye (e.g., activated charcoal) may be present in any suitable amount, such as from 0.1 to 5 wt. %, e.g., 0.3 to 1 wt. %.

The microporous membrane may enclose both the primary reservoir portion and at least one secondary reservoir portions. Thus, after activation, the membrane may be configured to allow evaporation of volatile material present in both the primary reservoir portion and at least one secondary reservoir portion.

### Volatile Material

The delivery engine comprises a volatile material.

The term “volatile material” as used herein, refers to a material that is vaporizable at room temperature and atmospheric pressure without the need of an energy source. The volatile material may be a composition comprised entirely of a single volatile material. The volatile material may also be mixture of volatile materials (i.e., the mixture has more than one volatile component). For the avoidance of doubt, the term “volatile material” as used herein includes compositions that comprise volatile components and non-volatile components. In other words, it is not necessary for all of the component materials of the volatile material to be volatile.

Examples of suitable non-volatile components that may be present in the volatile material include dyes, UV stabilizers, and antioxidants. In general, non-volatile components may be present in an amount of less than about 10%, such as less than about 5%. Any suitable volatile material in any amount or form, including a liquid or emulsion, may be used. In some embodiments of the invention, the volatile material may be a liquid.

When the volatile material is a liquid or emulsion, it may preferably be provided in the primary reservoir portion in free flowing form, preferably without being absorbed into a substrate or porous material. In particular, the volatile material may be provided in free liquid form, without being absorbed into a substrate or porous material (other than an amount of volatile that may be absorbed into a membrane, if the volatile material is provided in contact with a membrane). For example, greater than 50%, greater than 60%, greater than 70%, greater than 80%, greater than 90%, or greater than 95% of the volatile material may be provided in free liquid form or in a form that is not absorbed into a substrate or porous material.

Since the volatile material may include a portion of non-volatile material, liquids suitable for use in the volatile material may also include non-volatile components, such as carrier materials (e.g., water, solvents, etc). It should also be understood that when a volatile material (e.g., a liquid) is described herein as being “evaporated”, “volatilized”, “delivered”, “emitted”, or “released,” this refers to the volatilization of volatile component(s) of the volatile material, and does not require that any or all non-volatile components be emitted.

The volatile material can be in the form of perfume oil. Many conventional fragrance materials are volatile essential oils. The volatile material can be a volatile organic compound commonly available from perfumery suppliers. Furthermore, the volatile material can be synthetically or naturally formed materials. Examples include, but are not limited to: oil of bergamot, bitter orange, lemon, mandarin, caraway, cedar leaf, clove leaf, cedar wood, geranium, lavender, orange, origanum, petitgrain, white cedar, patchouli, neroili, rose absolute, and the like. In the case of air freshener or fragrances, the different volatile materials can be similar, related, complementary, or contrasting.

The volatile material may have a combined vapour pressure of at least 8 Pa at 25° C., such as at least 30 Pa at 25° C.

The volatile material may further include an anti-malodor composition capable of neutralising or reducing odors. Suitable anti-malodor compositions include cyclodextrin, reactive aldehydes, reactive ketones and ionones, particularly reactive aldehydes and ketones.

While not wishing to be bound by theory, the continuous delivery of a volatile material may be a function of various factors including membrane pore size; membrane surface area; the physical properties of a volatile material, such as molecular weight and saturation vapor pressure (“VP”); and the viscosity and/or surface tension of the composition containing the volatile material.

The composition may be formulated such that the composition comprises a volatile material mixture comprising about 10% to about 100%, by total weight, of volatile materials that each having a VP at 25° C. of less than about 0.01 torr; alternatively about 40% to about 100%, by total weight, of volatile materials each having a VP at 25° C. of less than about 0.1 torr; alternatively about 50% to about 100%, by total weight, of volatile materials each having a VP at 25° C. of less than about 0.1 torr; alternatively about 90% to about 100%, by total weight, of volatile materials each having a VP at 25° C. of less than about 0.3 torr. In one embodiment, the volatile material mixture may include 0% to about 15%, by total weight, of volatile materials each having a VP at 25° C. of about 0.004 torr to about 0.035 torr; and 0% to about 25%, by total weight, of volatile materials each having a VP at 25° C. of about 0.1 torr to about 0.325 torr; and about 65% to about 100%, by total weight, of volatile materials each having a VP at 25° C. of about 0.035 torr to about 0.1 torr. One source for obtaining the saturation vapor pressure of a volatile material is EPI Suite™, version 4.0, available from U.S. Environmental Protection Agency.

Two exemplary compositions comprising a volatile material mixture having volatile materials of varying VPs are set forth below in Tables 1 and 2. These compositions are shown by way of illustration and are not intended to be in any way limiting of the invention.

TABLE 1

| Wt % | Low VP (torr) | High VP (torr) |
|---|---|---|
| 27.71 | 0.175 | 0.325 |
| 20.78 | 0.0875 | 0.1125 |
| 13.86 | 0.0625 | 0.0875 |
| 8.66 | 0.0375 | 0.0625 |
| 8.66 | 0.0175 | 0.0325 |
| 6.93 | 0.00875 | 0.01125 |
| 6.93 | 0.00625 | 0.00875 |
| 3.18 | 0.00375 | 0.00625 |
| 1.27 | 0.00175 | 0.00325 |
| 0.95 | 0.000875 | 0.001125 |
| 0.64 | 0.000625 | 0.000875 |
| 0.32 | 0.000375 | 0.000625 |
| 0.09 | 0.000175 | 0.000325 |

TABLE 2

| Wt % | Low VP (torr) | High VP (torr) |
|---|---|---|
| 33.38 | 0.175 | 0.325 |
| 25.75 | 0.0875 | 0.1126 |
| 19.07 | 0.0625 | 0.0875 |
| 13.86 | 0.0375 | 0.0625 |
| 4.00 | 0.0175 | 0.0325 |
| 1.50 | 0.00875 | 0.01125 |
| 0.50 | 0.00625 | 0.00875 |
| 0.72 | 0.00375 | 0.00625 |
| 0.55 | 0.00175 | 0.00325 |
| 0.27 | 0.000875 | 0.001125 |
| 0.20 | 0.000625 | 0.000875 |
| 0.13 | 0.000375 | 0.000625 |
| 0.07 | 0.000175 | 0.000325 |

The viscosity of a volatile material may control how and when a volatile material is delivered to the microporous membrane. For example, less viscous compositions may flow faster than the more viscous volatile materials. Thus, the membrane may be first wetted with the less viscous materials. To help prevent liquid from seeping through the microporous membrane, volatile materials may have viscosities less than about 23 cP and surface tension less than about 33 mN/m.

In one embodiment, the composition containing a volatile material may have a viscosity of about 1.0 cP to less than about 25 cP, alternatively about 1.0 cP to less than about 23 cP, alternatively about 1.0 cP to less than about 15 cP.

The composition containing a volatile material may be designed such that the composition may include a surface tension of about 19 mN/m to less than about 33 mN/m, alternatively about 19 mN/m to less than about 30 mN/m, alternatively about 19 mN/m to less than about 27 mN/m.

The volatile material and surface area of the microporous membrane may be selected such that the delivery engine is configured to release the volatile material to a surrounding environment under ambient conditions and without an artificially generated airflow. For example, the delivery engine may be configured to release the volatile material over a desired period (e.g., 8 weeks) when placed in a still environment such as a small room or cupboard. As will be appreciated by a person skilled in the art, a delivery engine intended for use in still environments may include a larger membrane surface area, and higher volatility volatile material, than a product that is intended to be subjected to an artificial airflow.

### Activation of the Delivery Engine

As described herein, the volatile material contained within the delivery engine is configured to evaporate after an activation step is performed.

Thus, the delivery engine may be a delivery engine from which the volatile material cannot initially evaporate, but which may be activated to allow evaporation of the volatile material. In other words, the volatile material may be configured to evaporate from the delivery engine only after an activation step is performed. Thus, the volatile material may be configured to evaporate from the primary reservoir portion only after an activation step is performed.

As used herein, and unless prohibited by context, when a delivery engine is described as allowing the evaporation of volatile material, this is to be understood as covering a delivery engine that is in a state in which volatile material may evaporate (e.g., is activated), and also as covering an activatable delivery engine from which volatile material cannot currently evaporate but which may be activated to a state in which volatile material may evaporate.

Similarly, when a delivery engine is described as comprising a conduit for fluidly connecting the primary reservoir portion and at least one secondary reservoir portion, this is to be understood as covering a delivery engine in which the primary reservoir portion and at least one secondary reservoir portion are fluidly connected by the conduit, and also where the primary and secondary reservoir portions are not fluidly connected, but will become fluidly connected once an activation step is conducted.

Typically, the delivery engine may comprise an impermeable barrier enclosing the volatile material, which impermeable barrier may be removed or ruptured to allow the volatile material to evaporate. In such cases, the activation step referred to herein comprises removing or rupturing the impermeable barrier.

An example of a removable impermeable barrier is an impermeable film, foil, or laminate, such as a flexible (e.g., polymeric) film, a flexible (e.g., metal) foil, or a composite material (e.g., a foil/polymeric film laminate). The impermeable film, foil or laminate may be provided adhered to the delivery engine to prevent evaporation of volatile material, and may be peeled away or otherwise removed from the delivery engine in order to activate it and allow evaporation of the volatile material.

An example of a rupturable impermeable barrier is a rupturable substrate, such as a flexible (e.g., polymeric) film, a flexible (e.g., metal) foil, or a composite material (e.g., a foil/polymeric film laminate). The rupturable substrate may be provided inside or outside of the primary (and optionally secondary) reservoir portion, provided that the rupturable substrate, when in unruptured form, prevents evaporation of the volatile material. The rupturable substrate may be ruptured to allow evaporation of the volatile material, e.g., by actuation of a rupture element configured to rupture the rupturable substrate.

For example, in some embodiments of the invention in which the delivery engine comprises a membrane enclosing the reservoir, the delivery engine may further comprise a removable impermeable laminate (e.g., a plastic film or metal foil) covering the membrane. The removable impermeable laminate serves to prevent evaporation of the volatile material from the membrane before it is removed. The removable impermeable laminate may typically be removed by peeling off the removable impermeable laminate to expose the membrane to the surrounding environment, thereby allowing evaporation of the volatile material.

In alternative embodiments of the invention in which the delivery engine comprises a membrane enclosing the reservoir, the delivery engine may further comprise:
a rupturable substrate secured to said reservoir; and
a rupture element positioned adjacent to said rupturable substrate,
   wherein the microporous membrane encloses said rupturable substrate and said rupture element.

In such embodiments, the rupturable substrate serves to prevent contact between the volatile material and microporous membrane before the delivery engine is desired to be used. The rupturable substrate may be ruptured by actuating a rupture element, and such rupturing of the rupturable substrate will allow volatile material to flow through the rupturable substrate and contact the microporous membrane. The configuration of the rupturable substrate and rupture element is described in more detail hereinbelow.

The rupturable substrate may be located such that the primary reservoir portion and one or more secondary reservoir portions are fluidly isolated by the rupturable substrate before rupturing (e.g., the rupturable substrate may enclose the primary reservoir portion but not the secondary reservoir portion, with the volatile material being initially provided in the primary reservoir portion). Alternatively, the rupturable substrate may be located such that the primary and secondary reservoir portions are in fluid communication before rupturing of the rupturable substrate (e.g., the rupturable substrate may enclose both the primary and secondary reservoir portions). In either case, the rupturable substrate prevents the volatile material from contacting the microporous membrane before the rupturable substrate is ruptured.

The rupturable substrate may be ruptured by actuation of a rupture element. The rupture element may take any suitable form, for example an element that, on actuation, is configured to pierce the rupturable substrate, or is configured to impart a force onto another element that will pierce the rupturable substrate. The rupture element may be actuated by any appropriate means, such as push buttons, levers and hinges that may be incorporated into a housing of an apparatus, or by protrusions present in the housing of an apparatus, which protrusions may actuate the rupture element upon closing of the housing.

Delivery engines comprising rupturable substrates and rupture elements are described in more detail in U.S. Pat. Nos. 10,561,754, 10,561,755 and 10,561,756.

### Size of the Delivery Engine

As described herein, the delivery engine of the invention may have any appropriate size. In particular embodiments of the invention, the delivery engine may have the sizes discussed below.

A delivery engine for delivering a volatile material, the delivery engine comprising:
a primary reservoir portion containing a volatile material, where the volatile material is configured to evaporate from the primary reservoir portion after an activation step is performed; and
at least one secondary reservoir portion;
   wherein when the delivery engine is oriented for use:
at least one secondary reservoir portion is gravitationally below the primary reservoir portion; and
a total volume of the at least one secondary reservoir portion that is gravitationally below the primary reservoir portion is at least 0.24 mL, optionally at least 0.3 mL, more optionally at least 0.5 mL.

Such delivery engines may comprise an initial amount of volatile material (i.e., before activation) of from about 4 mL to about 10 mL, such as about 5 mL to about 9 mL, e.g., about 6 mL to about 7.5 mL.

Such delivery engines may comprise a microporous membrane as described hereinabove, where the microporous membrane has an (evaporative) surface area as described herein, such as from 20 cm² to 35 cm².

### Apparatus

The invention provides an apparatus comprising the delivery engine described herein. More specifically, the invention provides an apparatus for delivering a volatile composition, the apparatus comprising:
a housing having a front part, a back part; and
a delivery engine located within the housing, the delivery engine comprising:
a primary reservoir portion containing a volatile material, where the volatile material is configured to evaporate from the primary reservoir portion or where the volatile material is configured to evaporate from the primary reservoir portion after an activation step is performed; and
at least one secondary reservoir portion;
   wherein the housing is configured such that when the apparatus is oriented for use:
at least one secondary reservoir portion of the delivery engine is gravitationally below the primary reservoir portion of the delivery engine;
the primary reservoir portion of the delivery engine is visible through the front part of the housing, and one or both of (i) and (ii) apply:
(i) the at least one secondary reservoir portion that is gravitationally below the primary reservoir portion is at least partially obscured by the front part of the housing;
(ii) the at least one secondary reservoir portion that is gravitationally below the primary reservoir portion is formed from an opaque material.

For the avoidance of doubt, any feature of the delivery engine of the invention described above may apply equally to the delivery engine present of the apparatus of the invention. Where a feature of the delivery engine is described herein in relation to the delivery engine before activation, a corresponding feature may apply to the apparatus of the invention whether before, or after, such an activation.

Thus, the invention also provides an apparatus described above containing a delivery engine according to the invention, which delivery engine is in an activated or non-activated state.

The housing of the apparatus of the invention may be openable, such that once the delivery engine has reached end of life, it may be replaced with a new delivery engine. The housing may comprise a locking mechanism for releasably closing the housing.

In the apparatus of the invention, the primary reservoir portion of the delivery engine is typically visible through the front part of the housing, which allows a user to easily visualise a remaining level of volatile material. The primary reservoir portion may be visible through a window portion of the housing, and/or may be received by a window portion of the housing. As used herein, a window portion may refer to a cut-out portion of the housing, or to a transparent portion of the housing. However, the apparatus may reach end of life before all of the volatile material has evaporated. The apparatus of the invention advantageously ensures that when a small amount of volatile material remains, this is held in a secondary reservoir portion and so is generally not visible in the primary reservoir. Such secondary reservoir portion may be at least partially obscured by the housing (e.g., not visible through the housing), thereby hiding the residual amount of volatile material. This reduces the chance of users erroneously believing that an apparatus is not at end of life.

Although the secondary reservoir portion containing volatile material remaining at end of life is preferably not visible to a user, a part of the secondary reservoir portion(s) may be visible without significantly reducing the benefit provided by the invention. Therefore, less than 80%, less than 70%, less than 60%, less than 50%, less than 40%, less than 30%, less than 20% or less than 10% of the at least one secondary reservoir portion(s) may be visible through the front part of the housing.

Alternatively, or in addition to the at least partial obscuring of the at least one secondary reservoir portion by the front part of the housing, the at least one secondary reservoir portion may be formed from an opaque material. This ensures that volatile material present in any secondary reservoir portions is not visible.

The housing is configured such that when the apparatus is in use, the delivery engine is oriented such that at least one secondary reservoir portion is gravitationally below the primary reservoir portion. This is to be understood as meaning that at least one secondary reservoir portion is gravitationally below the primary reservoir portion when the apparatus is oriented for normal use, such as standing on a flat surface or adhered to a wall. This has the effect that when the apparatus is in use and reaches end of life, the volatile material is present within at least one secondary reservoir portion, and the primary reservoir portion appears (substantially) empty.

For example, the housing may comprise a base portion that is configured to rest on a flat surface. In this case, when the base portion is resting on a flat surface, the delivery engine will be oriented such that at least one secondary reservoir portion is gravitationally below the primary reservoir portion. In such cases, the base portion may be formed from the front part, back part, a different part of the housing, or combinations thereof. Alternatively, the housing may comprise an attachment means for adhering or otherwise attaching/suspending the apparatus to or from a wall or other object/surface, wherein when the housing is attached or suspended as intended, at least one secondary reservoir portion is gravitationally below the primary reservoir portion. An example of a suitable attachment means is an adhesive strip, or an aperture configured to receive a hook that is attached to a wall or ceiling. A skilled person will appreciate that other attachment means may be used, and the invention is not limited to the attachment means explicitly described herein.

In order to facilitate evaporation of the volatile material, the housing may comprise a plurality of apertures to allow volatile material to pass from the delivery engine to a surrounding environment. It may be desirable for such apertures to be located on a back part of the housing, since this might provide an improves aesthetic effect for the front part of the housing. Thus, the back part of the housing may comprise a plurality of apertures for exposing an evaporative portion of the delivery engine. For example, when the delivery engine comprises a membrane enclosing the primary and secondary reservoir portions, the membrane being configured to allow evaporation of liquid volatile material, the membrane may serve as the evaporative portion of the delivery engine. In some embodiments of the invention, the plurality of apertures may expose from about 40% to about 90% of the surface area of the evaporative portion (e.g., membrane) of the delivery engine. In certain embodiments of the invention, at least about 60% of the total surface area of the plurality of apertures may results from individual apertures of said plurality that each have an area of at least 30 mm² and an aspect ratio of at least about 1:2. Suitable aperture sizes, shapes and evaporative portion exposure percentages are described in more detail in U.S. Pat. No. 11,207,440.

Purely by way of illustration, the invention is described in more detail below with reference to the Figures.

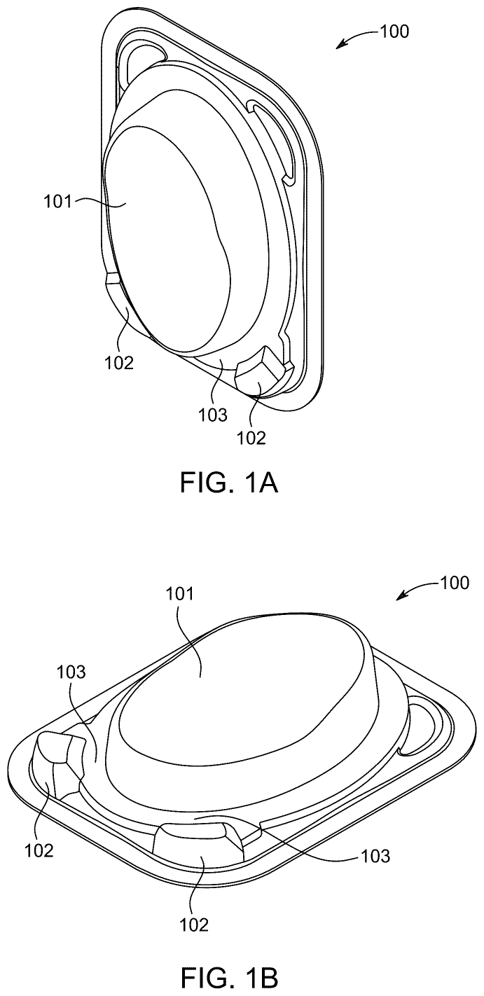

FIGS. 1A and 1B show two views of a delivery engine 100, comprising a primary reservoir portion 101 and secondary reservoir portions 102. The primary reservoir portion 101 contains a volatile material, which volatile material is configured to evaporate from the primary reservoir portion 101 after an activation step is performed. A conduit region 103 surrounding the primary reservoir portion 101 acts as a conduit to fluidly connect the primary reservoir portion 101 to the secondary reservoir portions 102. The primary reservoir portion 101 and secondary reservoir portions 102 are enclosed by a microporous membrane (not shown) located on the reverse side of the delivery engine 100. The microporous membrane is configured to allow evaporation of volatile material contacting the membrane.

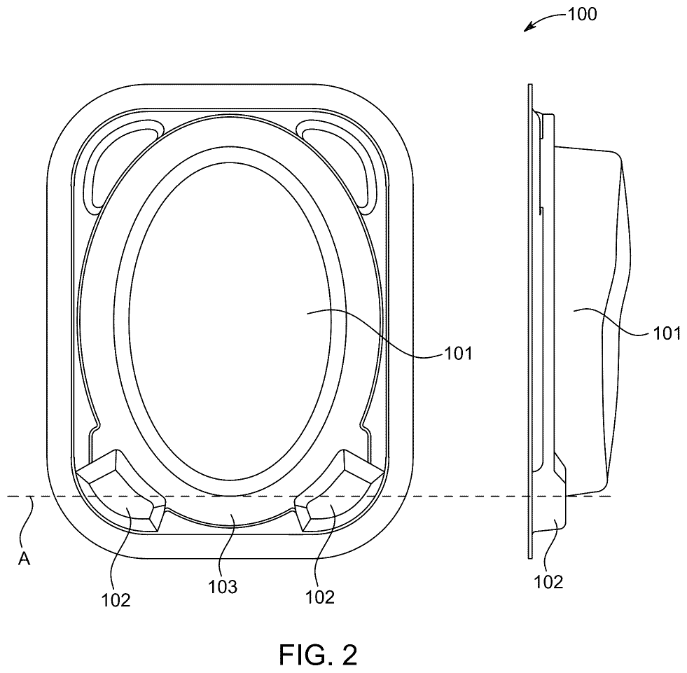

FIG. 2 shows a front and side view of the delivery engine 100. Dashed line A indicates the lowest point of the primary reservoir portion 101, such that when the delivery engine 100 is oriented for use, the components of the delivery engine 100 that are below the dashed line A are gravitationally below the primary reservoir portion 101. Thus, when oriented for use, the total volume of the secondary reservoir portions 102, plus the volume of the conduit region 103 that is below the dashed line A is at least 4% of an initial volume of volatile material. As described herein, the volume of the at least one secondary reservoir portion that is gravitationally below the primary reservoir portion is at least 4% of the initial volume of volatile material, and for the purposes of calculating this percentage, the volume of secondary reservoir portion is taken to include the volume of any conduit that is gravitationally below the primary reservoir portion and connects the primary reservoir portion to the secondary reservoir portion. Thus, for the delivery engine 100 shown in FIG. 2, the volume of the secondary reservoir portions 102, and conduit region 103 that is below the dashed line A is at least 4% of an initial volume of volatile material.

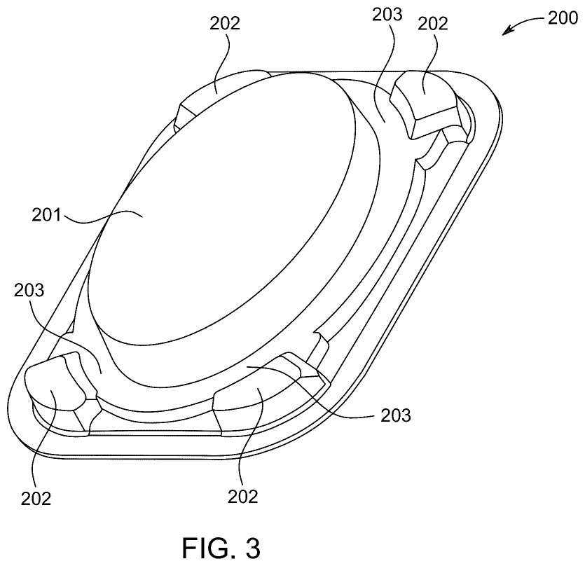

FIG. 3 shows an alternative delivery engine 200, comprising a primary reservoir portion 201, and secondary reservoir portions 202 at each of two opposing ends of the primary reservoir portion 201. As in FIG. 1, the delivery engine 200 comprises a conduit region 203 surrounding the primary reservoir portion 201, that acts as a conduit to fluidly connect the primary reservoir portion 201 to the secondary reservoir portions 202. The primary reservoir portion 201 and secondary reservoir portions 202 are enclosed by a microporous membrane (not shown) located on the reverse side of the delivery engine 200. The microporous membrane is configured to allow evaporation of volatile material contacting the microporous membrane. The delivery engine 200 of FIG. 3 may be inserted into a housing in more than one orientation, whilst still ensuring that secondary reservoir portions 202 are gravitationally below the primary reservoir portion 201. This improves ease of use for a user.

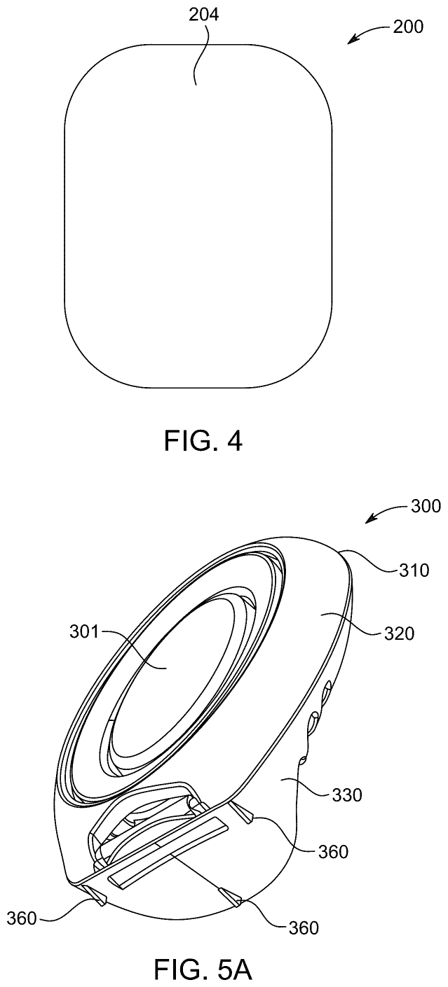

FIG. 4 shows a rear view of a delivery engine 200 according to the invention. The delivery engine 200 comprises a microporous membrane 204 that encloses the primary and secondary reservoir portions (not shown), which microporous membrane 204 is configured to allow evaporation of volatile material contacting the microporous membrane 204. In the delivery engine 200 shown in FIG. 4, the entire back surface of the delivery engine 200 is composed of the microporous membrane 204. Although FIG. 4 is described in relation to the delivery engine 200, the features shown and described in relation to FIG. 4 may apply equally to the delivery engine 100 shown in FIGS. 1A, 1B and 2.

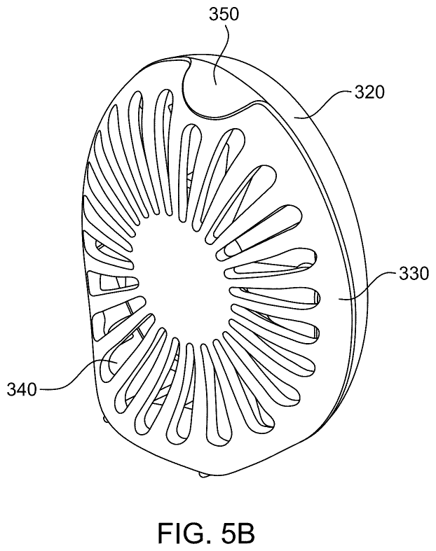

FIG. 5A shows an apparatus 300 for delivering a volatile composition. The apparatus 300 comprises a housing 310 having front part 320, and a back part 330. The apparatus further comprises a delivery engine as disclosed herein located within the housing 310. The primary reservoir portion 301 of the delivery engine is visible through the front part 320 of the housing 310, and is depicted being received by a window portion of the housing. However, other parts of the delivery engine are not visible through the front part 320 of the housing 310. As a result, the secondary reservoir portions are obscured by the front part 320 of the housing 310. FIG. 5B shows a more complete view of the back part 330 of the housing 310, which comprises a plurality of apertures 340 for exposing an evaporative portion (e.g., a microporous membrane) of the delivery engine. The housing 310 is openable so that the delivery engine may be replaced once it has reached end of life, and the back part 330 also comprises a locking mechanism 350 for releasably closing the housing. However, the apparatus of the invention is not limited to those comprising openable housings.

When the housing of FIGS. 5A and 5B is oriented for use (e.g., when base portions 360 are placed on a flat surface), at least one secondary reservoir portion of the delivery engine is gravitationally below the primary reservoir portion of the delivery engine.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A delivery engine for delivering a volatile material, the delivery engine comprising:
a primary reservoir portion containing an initial volume of the volatile material, wherein the volatile material is configured to evaporate from the primary reservoir portion after an activation step is performed; and
at least one secondary reservoir portion;
wherein when the delivery engine is oriented for use:
at least one secondary reservoir portion is gravitationally below the primary reservoir portion; and
a total volume of the at least one secondary reservoir portion that is gravitationally below the primary reservoir portion is at least 4% of the initial volume of volatile material.

2. The delivery engine according to claim 1, wherein when the delivery engine is oriented for use, a total volume of the at least one secondary reservoir portion that is gravitationally below the primary reservoir portion is from 4% to 30% of the initial volume of volatile material.

3. The delivery engine according to claim 1, wherein either (a) or (b) applies:
(a) the primary reservoir portion and at least one secondary reservoir portion are in fluid communication, or
(b) the delivery engine is configured such that after an activation step is performed, the primary reservoir portion and at least one secondary reservoir portion are in fluid communication.

4. The delivery engine according to claim 3, wherein:
when:
the delivery engine is oriented such that at least one secondary reservoir portion is gravitationally below the primary reservoir portion, and
the primary reservoir portion is in fluid communication with at least one secondary reservoir portion,
then the at least one secondary reservoir portion is able to hold a volume of volatile material that is at least 4% of the initial volume of volatile material in a state that is gravitationally below the primary reservoir.

5. The delivery engine according to claim 1, wherein the delivery engine is a single-use delivery engine and/or wherein the delivery engine is not refillable.

6. The delivery engine according to claim 1, wherein the volatile material is configured to simultaneously evaporate from both the primary reservoir portion and the at least one secondary reservoir portion after an activation step is performed.

7. The delivery engine according to claim 1, wherein the delivery engine comprises a microporous membrane enclosing the primary and secondary reservoir portions, where the microporous membrane is configured to allow evaporation of volatile material contacting the microporous membrane.

8. The delivery engine according to claim 7, wherein after an activation step is performed, the microporous membrane is configured to allow evaporation of volatile material present in both the primary reservoir portion and at least one secondary reservoir portion.

9. The delivery engine according to claim 1, wherein the delivery engine is for use in a housing, and the primary reservoir portion is configured to be received by a window portion of the housing.

10. The delivery engine according to claim 1, wherein the primary reservoir portion is formed from a transparent or translucent material.

11. The delivery engine according to claim 1, wherein the delivery engine is for use in a housing, and one or both of (i) and (ii) apply:
(i) the at least one secondary reservoir portion is configured to be at least partially obscured by the housing;
(ii) the at least one secondary reservoir portion is formed from an opaque material.

12. The delivery engine according to claim 1, wherein the delivery engine comprises at least one secondary reservoir portion at each of two opposing ends of the primary reservoir portion.

13. The delivery engine according to claim 1, wherein the delivery engine is configured to release a volatile material to a surrounding environment without an artificially generated airflow.

14. A delivery engine for delivering a volatile material, the delivery engine comprising:
a primary reservoir portion containing a volatile material, where the volatile material is configured to evaporate from the primary reservoir portion after an activation step is performed; and
at least one secondary reservoir portion;
wherein when the delivery engine is oriented for use:
at least one secondary reservoir portion is gravitationally below the primary reservoir portion; and
a total volume of the at least one secondary reservoir portion that is gravitationally below the primary reservoir portion is at least 0.24 mL.

15. An apparatus for delivering a volatile composition, the apparatus comprising:
a housing having a front part, a back part; and
a delivery engine located within the housing, the delivery engine comprising:
a primary reservoir portion containing a volatile material, where the volatile material is configured to evaporate from the primary reservoir portion or where the volatile material is configured to evaporate from the primary reservoir portion after an activation step is performed; and
at least one secondary reservoir portion;
wherein the housing is configured such that when the apparatus is oriented for use:
at least one secondary reservoir portion of the delivery engine is gravitationally below the primary reservoir portion of the delivery engine;
the primary reservoir portion of the delivery engine is visible through the front part of the housing, and one or both of (i) and (ii) apply:
(i) the at least one secondary reservoir portion that is gravitationally below the primary reservoir portion is at least partially obscured by the front part of the housing;
(ii) the at least one secondary reservoir portion that is gravitationally below the primary reservoir portion is formed from an opaque material.

16. The apparatus according to claim 15, wherein the back part of the housing comprises a plurality of apertures for exposing an evaporative portion of the delivery engine.

17. The apparatus according to claim 16, wherein the delivery engine comprises a microporous membrane enclosing the primary and secondary reservoir portions, where the microporous membrane is configured to allow evaporation of liquid volatile material, and where the evaporative portion of the delivery engine is the microporous membrane.

18. The apparatus according to claim 15, wherein the housing is openable and the delivery engine is replaceable.

19. The apparatus according to claim 18, wherein the housing comprises a releasable locking mechanism for releasably closing the housing.

20. The apparatus according to claim 15, wherein when the apparatus is oriented for use a total volume of the at least one secondary reservoir portions that are gravitationally below the primary reservoir portion is at least 4% of an initial volume of volatile material.
